(19) United States
(12) Patent Application Publication
(10) Pub. No.: US 2007/0122827 A1
(43) Pub. Date: May 31, 2007

(54) Target nucleic acid signal detection

(75) Inventors: Joseph A. Sorge, Del Mar, CA (US); Carsten-Peter Carstens, San Diego, CA (US); Craig Robert Monell, La Jolla, CA (US); Alexander Belyaev, San Diego, CA (US)

Correspondence Address: PALMER & DODGE, LLP;KATHLEEN M. WILLIAMS / STR, 111 HUNTINGTON AVENUE, BOSTON, MA, 02199, US

(73) Assignee: Stratagene California

(21) Appl. No.: 11/546,675

(22) Filed: Oct. 11, 2006

Related U.S. Application Data
(60) Provisional application No. 60/725,916, filed on Oct. 11, 2005.

(51) Int. Cl. C12Q 1/68; C12P 19/34

(52) U.S. Cl. 435/6; 435/91.2

(57) ABSTRACT

Provided herein are methods, compositions and kits for detecting a target nucleic acid. A target nucleic acid can be produced, for example, by a cleavage reaction in an assay for detection of biological samples. In one aspect, a described method comprises the following steps: hybridizing the target nucleic acid having a 5′ end and a 3′ end to a probe to form a circular hybridization complex; forming a covalent circular target nucleic acid using the probe as a template for nucleic acid synthesis; and detecting the covalent circular target nucleic acid.

### RELATED APPLICATIONS

The application claims priority to U.S. Provisional Application No. 60/725,916, filed Oct. 11, 2005. The entire teachings of the above application are incorporated herein by reference.

### BACKGROUND OF THE INVENTION

Techniques for polynucleotide detection have found widespread use in basic research, diagnostics, and forensics. Polynucleotide detection can be accomplished by a number of methods. Most methods rely on the use of the polymerase chain reaction (PCR) to amplify the amount of target nucleic acid. The sensitivity with which nucleic acids can be detected has resulted in the development of assays for the detection of non-nucleic acid biological samples. For example, several so called “proximity-probe” assays are known in the art. Proximity-probes produce a detectable signal when the probes bind an analyte within close proximity to each other. Such assays are described in U.S. Publication No. 2002/0064779 (Baez et al.), U.S. Pat. No. 6,511,809 (assigned to E.I. du Pont de Nemours and Company), U.S. Publication No. 2005/0003361 (Fredriksson, S.), PCT publication WO 2005/019470 (Aclara Biosciences, Inc.), and U.S. Publication No. 2005/0026180 (assigned to The Board of Trustees of Leland Stanford Junior University).

### SUMMARY OF THE INVENTION

Described herein are methods, reagents and kits for the detection of nucleic acids.

In one aspect, the invention provides a method for detecting a target nucleic acid, or a target nucleic acid. A target nucleic acid can be produced, for example, by a cleavage reaction in an assay for detection of biological samples. The method comprises the following steps:
hybridizing the target nucleic acid having a 5′ end and a 3′ end to a probe to form a circular hybridization complex;
forming a covalent circular target nucleic acid using the probe as a template for nucleic acid synthesis; and;
detecting the covalent circular target nucleic acid.

The probe has a 5′ end and a 3′ end and comprises a first target nucleic acid binding site and a second target nucleic acid binding site. The probe is blocked from its 3′ end and not ligatable from its 5′ end.

In another aspect, the present invention provides a composition for detecting a target nucleic acid. The composition comprises a probe, a polymerase, and optionally primers. The probe has a 5′ end and a 3′ end and comprises a first target nucleic acid binding site and a second target nucleic acid binding site. The probe is blocked from its 3′ end and not ligatable from its 5′ end.

In yet another aspect, a kit for detecting a target nucleic acid is provided. The kit comprises a probe, a polymerase, a primer and packaging material therefor.

### BRIEF DESCRIPTION OF THE DRAWINGS

FIG. 1 illustrates one embodiment of a target nucleic acid and a probe.

FIG. 2 shows an example of extension, ligation steps for the detection method.

FIG. 3 shows an example of a linear hybridization complex formed between one target nucleic acid and two probes.

FIG. 4 shows examples of simultaneously detecting multiple target nucleic acids

### DETAILED DESCRIPTION

Definitions

As used herein, the term “hybridization” is used in reference to the pairing of complementary (including partially complementary) polynucleotide strands. Hybridization and the strength of hybridization (i.e., the strength of the association between polynucleotide strands) is impacted by many factors well known in the art including the degree of complementarity between the polynucleotides, stringency of the conditions involved affected by such conditions as the concentration of salts, the melting temperature (Tm) of the formed hybrid, the presence of other components (e.g., the presence or absence of polyethylene glycol or formamide), the molarity of the hybridizing strands and the G:C content of the polynucleotide strands.

As used herein, “nucleic acid polymerase” or “polymerase” refers to an enzyme that catalyzes the polymerization of nucleoside triphosphates, and encompasses DNA polymerases, RNA polymerases, reverse transcriptases and the like. Generally, the enzyme will initiate synthesis at the 3′-end of the primer annealed to the target sequence, and will proceed in the 5′-direction along the template, and if possessing a 5′ to 3′ nuclease activity, hydrolyzing intervening, annealed probe to release both labeled and unlabeled probe fragments, until synthesis terminates. Known DNA polymerases include, for example, E. coli DNA polymerase I, T7 DNA polymerase, Thermus thermophilus (Tth) DNA polymerase, Bacillus stearothermophilus DNA polymerase, Thermococcus litoralis DNA polymerase, Thermus aquaticus (Taq) DNA polymerase and Pyrococcus furiosus (Pfi) DNA polymerase.

As used herein, a polymerase which is “exonuclease deficient” refers to a DNA polymerase having less than 10%, 5%, 1%, 0.5%, or 0.1% of the 5′ to 3′ exonuclease activity of a wild type enzyme. The phrase “exonuclease deficient” means having undetectable activity or having less than about 1%, 0.5%, or 0.1% of the 5′ to 3′ exonuclease activity of a wild type enzyme. 5′ to 3′ exonuclease activity may be measured by an exonuclease assay which includes the steps of cleaving a nicked substrate in the presence of an appropriate buffer, for example 10 mM Tris-HCl (pH 8.0), 10 mM MgCl₂ and 50 μg/ml bovine serum albumin) for 30 minutes at 60° C., terminating the cleavage reaction by the addition of 95% formamide containing 10 mM EDTA and 1 mg/ml bromophenol blue, and detecting nicked or un-nicked product.

As used herein, when one polynucleotide is said to “hybridize” to another polynucleotide, it means that there is some complementarity between the two polynucleotides or that the two polynucleotides form a hybrid under high stringency conditions.

As used herein, “Tₘ” and “melting temperature” are interchangeable terms which are the temperature at which 50% of a population of double-stranded polynucleotide molecules becomes dissociated into single strands. The equation for calculating the Tm of polynucleotides is well known in the art. For example, the Tₘ may be calculated by the following equation: Tₘ=69.3+0.41×(G+C)%−650/L, wherein L is the length of the probe in nucleotides. The Tₘ of a hybrid polynucleotide may also be estimated using a formula adopted from hybridization assays in 1 M salt, and commonly used for calculating Tₘ for PCR primers: [(number of A+T)×2° C.+(number of G+C)×4° C.], see, for example, C. R. Newton et al. PCR, 2^{nd} Ed., Springer-Verlag (New York: 1997), p. 24. Other more sophisticated computations exist in the art, which take structural as well as sequence characteristics into account for the calculation of Tₘ. A calculated Tₘ is merely an estimate; the optimum temperature is commonly determined empirically.

A “nucleic acid synthesis” reaction or a “chain elongation” reaction means a reaction between a target-probe hybrid and a nucleotide which results in the addition of the nucleotide to a 3′-end of the primer such that the incorporated nucleotide is complementary to the corresponding nucleotide of the target polynucleotide. Primer extension reagents typically include (i) a polymerase enzyme; (ii) a buffer; and (iii) one or more extendible nucleotides.

As used herein, “polymerase chain reaction” or “PCR” refers to an in vitro method for amplifying a specific polynucleotide template sequence. The PCR reaction involves a repetitive series of temperature cycles and is typically performed in a volume of 50-100 μl. The reaction mix comprises dNTPs (each of the four deoxynucleotides DATP, dCTP, dGTP, and dTTP), primers, buffers, DNA polymerase, and polynucleotide template. One PCR reaction may consist of 5 to 100 “cycles” of denaturation and synthesis of a polynucleotide molecule.

A “nucleotide analog”, as used herein, refers to a nucleotide in which the pentose sugar and/or one or more of the phosphate esters is replaced with its respective analog. Exemplary pentose sugar analogs are those previously described in conjunction with nucleoside analogs. Exemplary phosphate ester analogs include, but are not limited to, alkylphosphonates, methylphosphonates, phosphoramidates, phosphotriesters, phosphorothioates, phosphorodithioates, phosphoroselenoates, phosphorodiselenoates, phosphoroanilothioates, phosphoroanilidates, phosphoroamidates, boronophosphates, etc., including any associated counterions, if present. Also included within the definition of “nucleotide analog” are nucleobase monomers which can be polymerized into polynucleotide analogs in which the DNA/RNA phosphate ester and/or sugar phosphate ester backbone is replaced with a different type of linkage.

A “target nucleic acid,” as used herein, refers to a target nucleic acid whose presence is to be determined. A “target nucleic acid” can comprise deoxyribonucleic acid, ribonucleic acid or mixtures thereof. As used herein, a “target nucleic acid” has a defined 5′ end and 3′ end. In addition, a “target nucleic acid” can further comprise non-natural nucleic acids. Generally, a “target nucleic acid” is of a sufficient length to hybridize to two target nucleic acid binding sites of the probe, and is therefore generally at least 10 bases in length, typically at least 20 bases in length, for example, at least 25, 30, or 35 bases in length. While the target nucleic acid can be large nucleic acid fragments, it is generally limited to nucleic acids of 20 kilobases or less.

As used herein, a “covalent circular target nucleic acid” refers to a circular nucleic acid that is formed by nucleic acid synthesis using the probe as a template from the circular hybridization complex formed between a target nucleic acid and a probe. The “covalent target nucleic acid” forms as a result of ligating the original target nucleic acid with the nucleic acid synthesis product.

As used herein, a “probe” refers to a type of oligonucleotide having or containing a sequence which is complementary to another polynucleotide, e.g., a target nucleic acid. The probe of the present invention is generally between 50 and 300 bases in length, typically between 100 and 200 bases in length.

The probe of the present invention comprises two “target nucleic acid binding sites.” As used herein, a “target nucleic acid binding site” and “TNA binding site” refer to a region within the probe which is complementary to a portion of the target nucleic acid. For example, a “first target nucleic acid binding site” of the probe is complementary to and hybridizes to a “first probe interacting site” of the target nucleic acid. A “target nucleic acid binding site” is generally between 10 and 40 bases in length, typically between 15 and 25 bases in length.

A target nucleic acid binding site can be located at the “5′ end” or “3′ end” of the probe. As used herein, a target nucleic acid binding site, the most proximal nucleotide of which is located within 5 bases from the 5′ terminus of the probe, is said to be “at the 5′ end” of the probe. Similarly, a target nucleic acid binding site, the most proximal nucleotide of which is located within 5 bases from the 3′ terminus of the probe, is said to be “at the 3′ end” of the probe.

As used herein, a primer binding region is said to be “located at least a certain number of bases from one of the target nucleic acid binding sites” when the nucleotide closest to one of the target nucleic acid binding site is located at least a certain number of bases from that target nucleic acid binding site. For example, a primer binding region whose nucleotide closest to the first primer binding region is located at least 10 bases from the first primer binding region or whose nucleotide closest to the second primer binding region is said to be located at least 10 bases from the second target nucleic acid binding site.

As used herein, a “circular hybridization complex” refers to a hybrid complex that is formed between a target nucleic acid and a probe. The probe comprises two target nucleic acid binding sites each of which is complementary to and hybridizes with a different region within the target nucleic acid, such that a circular, partially double stranded structure, or a “circular hybridization complex” is formed.

As used herein, the term “primer binding site” refers to the complimentary sequence within the probe to which an oligonucleotide primer can hybridize.

Generally the 3′ terminus of the probe will be “blocked” to prohibit creation of an extension product. “Blocking” can be achieved by using non-complementary bases at or near the 3′ terminus, or by adding a chemical moiety such as biotin or a phosphate group to the 3′ hydroxyl of the last nucleotide. Blocking can also be achieved by removing the 3′-OH or by using a nucleotide that lacks a 3′-OH such as dideoxynucleotide, or by other methods known to one skilled in the art.

The 5′ terminus of the probe will generally be modified such that it is “not ligatable” or “non-ligatable.” To modify the probe to be “non ligatable”, the 5′ phosphoryl moiety can be removed or replaced, or an additional moiety can be attached to the 5′ phosphoryl moiety to prevent ligation with another nucleic acid, for example, by a ligase enzyme.

Target Nucleic Acid

The present invention contemplates the detection and/or quantitation of a nucleic acid.

The methods described herein can detect a very broad range of nucleic acids. In many circumstances, the nucleic acid can be detected directly; however, in other instances, the nucleic acid can be processed prior to the detection step. The target nucleic acid can be generated by any number of means. For example, it can be generated from a cleavage reaction by a restriction enzyme or other endo- or exonucleases. Alternatively, it can form as a result of a specific or non-specific cleavage of a longer nucleic acid strand, and can be generated enzymatically or chemically. The target nucleic acid of the present invention also contemplates fragments generated by aged tissue, apoptotic cells, or the consequence of any other natural, biological or chemical reaction that may generate nucleic acid fragments.

According to the present invention, a target nucleic acid is a nucleic acid whose presence needs to be determined, for example, in a sample. The target nucleic acid can be of any length greater than 10 bases in length, for example 20, 25, 30, 40, 50, 60, 100 bases in length or more. The target nucleic acid is typically single stranded. However, the presence of a double stranded nucleic acid can also readily be detected by first denaturing the sample, then using one of the two strands as a target nucleic acid for determination. Alternatively, if the double stranded nucleic acid whose presence is to be determined contains a single stranded region, this region can be used as sites of hybridization with the target nucleic acid binding sites of the probe.

The target nucleic acid can be composed of natural, non-natural nucleic acids or combinations thereof. The only requirement of the target nucleic acid is that the 3′ end of the target nucleic acid must support polymerase dependent extension reactions (i.e., must not be blocked), and that the 5′ end must be ligatable. Thus, while it is not necessary that the entire sequence of the target nucleic acid be known, the sequence of the 5′ end and 3′ end of the target nucleic acid must be known. The 5′ end and 3′ end of the target nucleic acid contain probe interacting sites which are complementary to the target nucleic acid binding sites of the probe.

In certain embodiments, the nucleic acid whose presence is to be determined is not ideal for detection using the methods described herein, for example, because one the ends is blocked or damaged or the nucleic acid is circular. It will be appreciated by one of skill in the art that such nucleic acids can be converted to a target nucleic acid by processing the nucleic acid either enzymatically or chemically. For example, a circular nucleic acid can be linearized using specific or non-specific nucleases at specified conditions, and blocked ends preventing extension can likewise be removed by limited treatment with exonucleases. In addition, it will also be apparent to one of skill in the art that detection of long nucleic acids (i.e., greater than 10 kb) can also be performed, for example, by generating a smaller fragment by restriction endonucleases.

Probe

According to the present invention, the probe has a 5′ end and a 3′ end. The probe further comprises a first target nucleic acid binding site and a second target nucleic acid binding site. The probe is blocked from its 3′ end. Furthermore, the probe is not ligatable from its 5′ end. The probe comprises two target nucleic acid binding sites, each capable of binding to a different region of the target nucleic acid. The probe is capable of forming a circular hybridization complex with the target nucleic acid.

The probe of the present invention can be of any length, so long as it is capable of forming a hybridization complex with the target nucleic acid (e.g., it must contain two target nucleic acid binding sites), and also allowing for effective extension reaction from the end of the target nucleic acid. In one embodiment, the probe is between 50 and 350 bases in length. In another embodiment, the probe is between 100 and 200 bases in length.

The probe can comprise natural or non-natural nucleic acids, or combinations thereof. The probe can comprise a nucleic acid analog or chimera comprising nucleic acid and nucleic acid analog monomer units, such as 2-aminoethylglycine, a peptide nucleic acid (PNA), or a locked nucleic acid (LNA). For example, part or all of the oligonucleotide may be LNA or a LNA/nucleic acid (DNA or RNA) chimera. In one embodiment, the oligonucleotide comprises at least one locked nucleic acid. Locked nucleic acids represent a class of conformationally restricted nucleotide analogues described, for example, in WO 99/14226, which is incorporated by reference. LNAs hybridize more strongly to both DNA and RNA than naturally occurring nucleotides. Oligonucleotides containing the locked nucleotide are described in Koshkin, A. A., et al., Tetrahedron (1998), 54: 3607-3630) and Obika, S. et al., Tetrahedron Lett. (1998), 39: 5401-5404), both of which are incorporated by reference. Introduction of a locked nucleotide into an oligonucleotide improves the affinity for complementary sequences and increases the melting temperature by several degrees (Braasch, D. A. and D. R. Corey, Chem. Biol. (2001), 8:1-7). The invention can be carried out with any of the LNAs known in the art, for example, those disclosed in WO 99/14226 and in Latorra D, et al., 2003. Hum. Mutat. 22: 79-85, both of which are incorporated herein by reference. More specific binding can be obtained and more stringent washing conditions can be employed using LNA analogs, with the advantage that the amount of background noise is reduced significantly.

The probe comprises a first target nucleic acid binding site and a second target nucleic acid binding site. Target nucleic acid binding sites are sequences within the probe which are complementary to a sequence within the target nucleic acid. The first and second target nucleic acid binding sequences are complementary to two different regions within the target nucleic acid. The target nucleic acid binding sites are generally between 10 and 40 bases in length. In one embodiment, the target nucleic acid binding site is between 15 and 25 bases in length. The target nucleic acid binding sequences can be positioned at the 3′ or 5′ termini (i.e., 3′ or 5′ ends). Alternatively, the target nucleic acid binding sites can be positioned anywhere within the probe, so long as the probe and the target nucleic acid can form a circular hybridization complex. In one embodiment, the first target nucleic acid binding site is at either of the 5′ end or 3′ end of the probe. In another embodiment, the second target nucleic acid binding site is located at the other of the 5′ end or 3′ end of the probe. In yet another embodiment, the first target nucleic acid binding site which hybridizes to a site at the 3′ end of the target nucleic acid is located at or near the 5′ end of the probe, and the second target nucleic acid binding site which hybridizes to a site at the 5′ end of the target nucleic acid is located at or near the 3′ end of the probe.

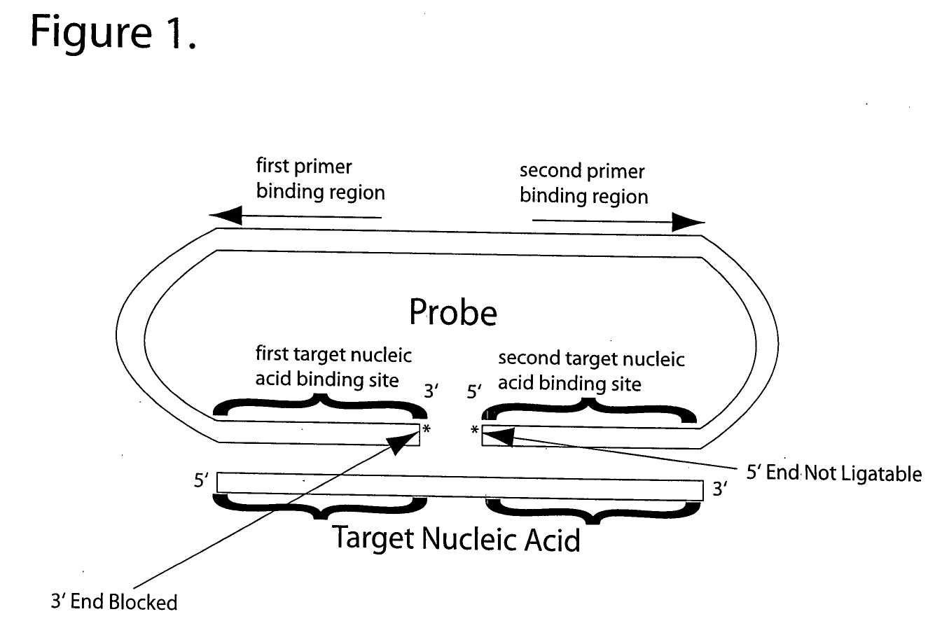

The probe is capable of forming a hybridization complex with the target nucleic acid. The hybridization complex is formed through hybridization of the target nucleic acid with the probe at the two target nucleic acid binding sites, each of which is complementary to different portions within the target nucleic acid. An example of a hybridization complex is shown in FIG. 1.

As described herein, the 5′ end of the probe is not ligatable. In one embodiment, the 5′ end of the probe is dephosphorylated. In another embodiment, a chemical moiety is attached to the 5′ end of the probe, either on the phosphoryl group or replacing the phosphoryl moiety, to prevent ligation. In yet another embodiment, the nucleotide at the 5′ end is a non-natural nucleic acid which does not permit ligation using a ligase enzyme.

The probe according to the present invention does not support an extension reaction from its 3′ end. In one embodiment, the 3′ end nucleotide of the probe is blocked. For example, the 3′ end nucleotide can be a dideoxy nucleic acid or any other nucleic acid (natural or otherwise) which does not allow extension using a polymerase enzyme. In another embodiment, a chemical moiety is attached to the 3′ end of the probe, for example, on the 3′ OH moiety, to prevent extension from that end. In still another embodiment, the 3′ end of the probe does not hybridize with a primer, such that the 3′ end of the probe cannot serve as a primer for an extension reaction by a polymerase.

In one embodiment, the probe also comprises a first primer binding region. In another embodiment, the probe further comprises a second primer binding region. In general, the primer binding region does not overlap with the target nucleic acid binding site of the probe. Furthermore, in embodiments encompassing a first primer binding region and a second primer binding region, the two primer binding regions generally do not overlap with each other. In one embodiment, the first primer binding region is located between the first and second target nucleic acid binding sequence of the probe. In another embodiment, the second primer binding region is located between the first and second target nucleic acid binding sequence of the probe. The primer binding region can be located at least 15 bases from one of the target nucleic acid binding sites, for example at least 20, 25, 30, 35, 40, 45 bases or more from one of the target nucleic acid binding sites. In still another embodiment, the probe comprises two primer binding regions, each of which is positioned between the first and second target nucleic acid binding region, and separated by at least 20 bases from one of the target nucleic acid binding sites. In another embodiment, they are separated by at least 30 bases from one of the target nucleic acid binding sites.

Detection of Target Nucleic Acids

As described herein, the method of detecting the target nucleic acid comprises the following steps:
hybridizing the target nucleic acid to a probe to form a circular hybridization complex;
forming a covalent circular target nucleic acid using the probe as a template for a nucleic acid synthesis; and
detecting the covalent circular target nucleic acid.

As previously described, the probe contains two target nucleic acid binding sites which are complementary to and hybridize to the target nucleic acid. The ideal Tₘ of the hybridization complex between the target nucleic acid and the probe is between 40° C. and 75° C., typically between 45° C. and 70° C., for example, between 50° C. and 65° C.

The equation for calculating the Tₘ of polynucleotides is well known in the art. For example, the Tₘ may be calculated by the following equation: Tₘ=69.3+0.41×(G+C)%−650/L, wherein L is the length of the oligonucleotide in nucleotides. The Tₘ of a hybrid polynucleotide may also be estimated using a formula adopted from hybridization assays in 1 M salt, and commonly used for calculating Tₘ for PCR primers: [(number of A+T)×2° C.+(number of G+C)×4° C.], see, for example, C. R. Newton et al. PCR, 2^{nd} Ed., Springer-Verlag (New York: 1997), p. 24. Other more sophisticated computations exist in the art, which take structural as well as sequence characteristics into account for the calculation of Tₘ. A calculated Tₘ is merely an estimate; the optimum temperature is commonly determined empirically. The stability and melting temperature of sequences can also be determined, for example, using programs such as mfold (Zuker (1989) Science, 244, 48-52) or Oligo 5.0 (Rychlik & Rhoads (1989) Nucleic Acids Res. 17, 8543-51). Methods for calculating the Tₘ of natural and non-natural nucleic acids are also known in the art. For example, the melting temperature LNA-DNA hybrids can be calculated using methods known in the art, for example as described in McTigue et al. (2004) Biochemistry, 43, 5388-5405, Tolstrup et al., (2003) Nucl. Acid Res. 31, 3758-62, incorporated by reference.

Extension

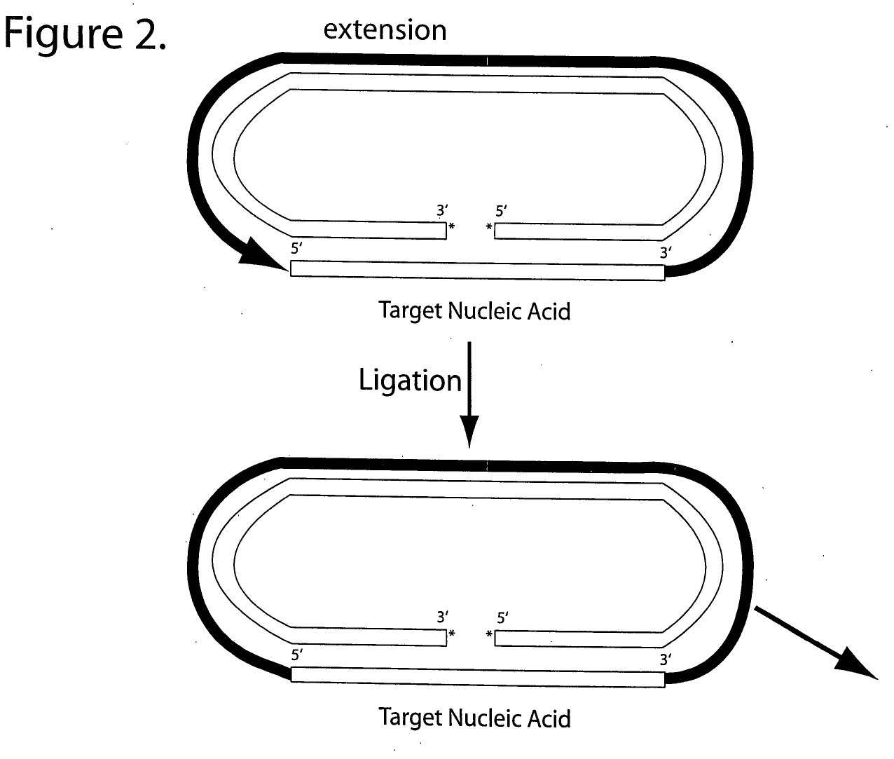

Once a circular hybridization complex is formed, the target nucleic acid is used as a primer in a template-dependent polymerization reaction. The extension step is carried out by providing a polymerase under conditions supporting polymerization (nucleotides, buffer, magnesium, and temperature appropriate for the given polymerase) known to one skilled in the art. The present invention contemplates the use of any polymerase known in the art. In one embodiment, the polymerase is a DNA polymerase. In another embodiment, the polymerase is an exonuclease deficient DNA polymerase. In still another embodiment, the polymerase is a thermostable DNA polymerase. The extension reaction is generally performed under conditions in which strand displacement does not occur. In one embodiment, a non strand-displacing DNA polymerase is used. In another embodiment, a DNA polymerase which is capable of strand-displacement is used under conditions in which strand displacement does not occur. For example, a thermostable enzyme such as Pfu DNA polymerase does not displace strands when extension is performed at temperatures of 65° C. or below. As previously described, the probe itself is incapable of serving as a primer for extension reactions, and is further not ligatable from its 5′ end. It merely serves as template for synthesis using the target nucleic acid as the primer (See, for example, FIG. 2).

Once extended (See, for example, FIG. 2), a covalent circular target nucleic acid is then formed by ligating the extension product to the 5′ end of the target nucleic acid.

According to methods described herein, a probe is provided in concentrations exceeding that of the target nucleic acid. The probe concentration can be at least 1.1 fold higher than the concentration of the target nucleic acid, for example at least 2, 3, 4, 5, 10, 20, 30, 50, 100 fold or more higher.

Detection of Covalent Circular Target Nucleic Acid

The covalent circular target nucleic acid is then detected. The covalent circular target nucleic acid can be detected using any method known in the art. In one embodiment, the covalent circular target nucleic acid is detected using the polymerase chain reaction (PCR). As previously described, the probe can contain one or more primer binding regions. PCR primers complementary to these sequences can be used.

The amount of covalent circular target nucleic acid can also be quantitated using PCR. Quantitative methods employing PCR, for example using fluorescence, real-time measurements, have been described in U.S. Pat. No. 5,723,591, U.S. Pat. No. 5,846,717, U.S. Pat. No. 5,994,056, U.S. Pat. No. 6,001,567, U.S. Pat. No. 6,348,314, and U.S. Pat. No. 6,635,427, which are incorporated by reference. Quantitative PCR (qPCR) is perhaps the most sensitive and flexible gene expression profiling method, which can be used to compare nucleic acid levels. In one embodiment, the covalent circular target nucleic acid is detected and quantitated using a TaqMan® assay (Applied Biosystems, Foster City, Calif.; See, for example, U.S. Pat. No. 5,723,591, which is incorporated herein by reference). The assay is performed during a PCR reaction. The TaqMan assay exploits the 5′-3′ exonuclease activity of DNA polymerases such as AMPLITAQ® DNA polymerase (Applied Biosystems, Foster City, Calif.). A fluorescent probe, specific for a given allele or mutation, is included in the PCR reaction. The fluorescent probe consists of an oligonucleotide with a 5′-reporter dye (e.g., a fluorescent dye) and a 3′-quencher dye. During PCR, if the fluorescent probe is bound to its target, the 5′-3′ nucleolytic activity of the polymerase cleaves the fluorescent probe between the reporter and the quencher dye. The separation of the reporter dye from the quencher dye results in an increase of fluorescence. The signal accumulates with each cycle of PCR and can be monitored with a fluorimeter. The covalent circular target nucleic acid can also be detected and quantitated using any method known to the skilled artisan, for example, as described in U.S. Pat. No. 5,538,848 which is incorporated herein by reference; polymerase chain reaction; branched hybridization methods (e.g., Chiron, U.S. Pat. Nos. 5,849,481, 5,710,264, 5,124,246, and 5,624,802, incorporated by reference); rolling circle replication (e.g., U.S. Pat. Nos. 6,210,884 and 6,183,960, incorporated by reference); NASBA (e.g., U.S. Pat. No. 5,409,818, incorporated by reference); molecular beacon technology (e.g., U.S. Pat. Nos. 6,277,607; 6,150,097; and 6,037,130, which are incorporated by reference); the adjacent probes method (U.S. Pat. No. 6,174,670B1, incorporated herein by reference in its entirety); Sunrise primer system (e.g., U.S. Pat. No. 5,866,336, incorporated by reference); Scorpion probe system (e.g., Thelwell et al., (2000) Nucleic Acids Research 28, 3752-3761; Whitcombe et al., (1999). Nature Biotechnology 17, 804-807, incorporated by reference); the Luminex/xMAP (microsphere) system; and the DzyNA-PCR system (e.g., U.S. Pat. No. 6,140,055, incorporated by reference).

Other Hybrid Complexes

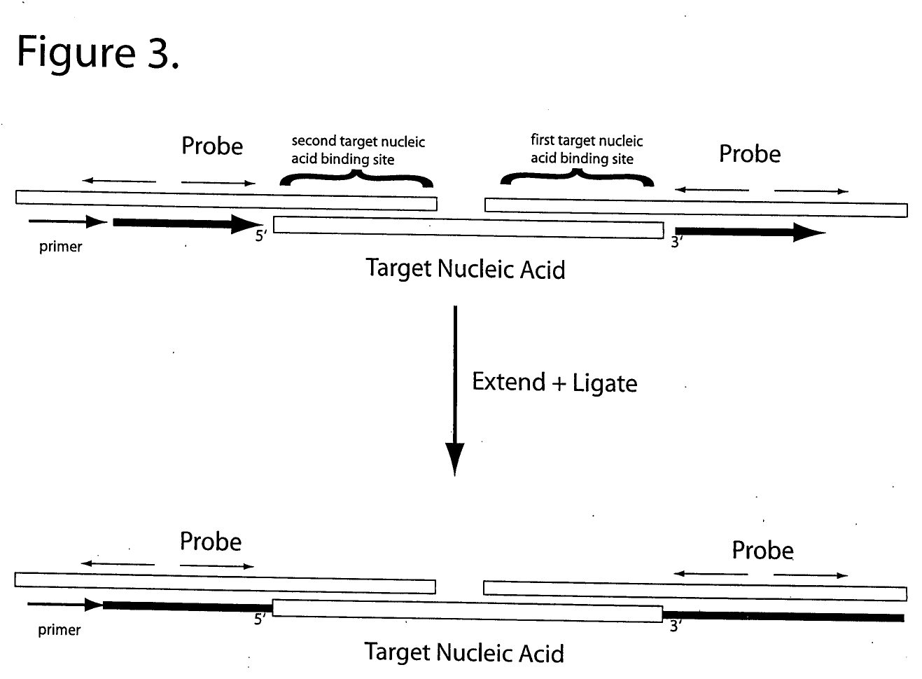

In rare cases, the excess concentration of the probe can result in formation of a linear hybridization complex, comprising one target nucleic acid and two probes, where the target nucleic acid hybridizes to first target nucleic acid binding site of the first probe and the second nucleic acid binding site of the second probe (See, for example, FIG. 3). Linear hybridization complexes such as this can be detected, for example, if extension reaction is performed in the presence of an additional primer, resulting in a double stranded complex.

Simultaneous Detection of Multiple Target Nucleic Acids

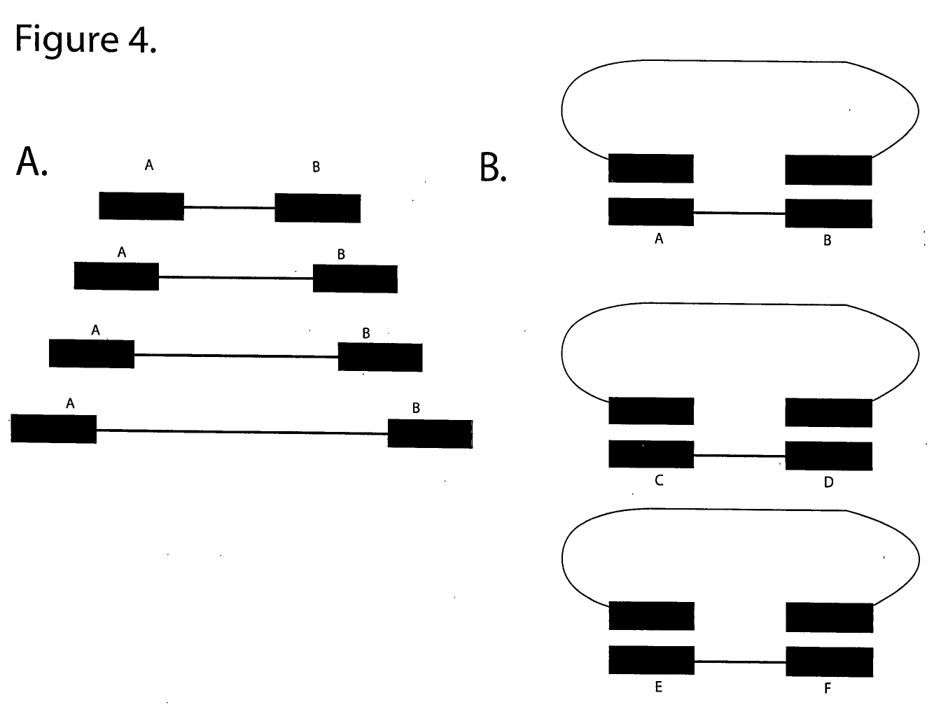

The methods described herein permit the detection of multiple target nucleic acids in a single reaction. In one embodiment, a plurality of target nucleic acids can be detected which can hybridize to the same probe (e.g., the target nucleic acids all having common first and second probe interacting sites), but have different lengths intervening the first and second probe interacting sites (See, for example, FIG. 4). For example, the targets can differ in the lengths intervening the first and second probe interacting sites by 50, 100, 150, 200, 250, 300, 400, 500 or more bases. Alternatively, the targets can differ by sequence. Each target may have a unique sequence which can be identified by sequence analysis, differential restriction digest patterns, or by polymerase chain reaction. The targets can also contain different aptamer sequences, with affinities to different antigens or chemical moieties.

In another embodiment, the plurality of target nucleic acids can differ in the probe interacting sites. For example, each probe interacts with a specific target nucleic acid (e.g., a unique probe for each target). Alternatively, a probe can interact with a subset of target nucleic acids. The first and second target nucleic acid binding sites of a probe can hybridize to a subset of 2, 3, 4, 5, 10, 20 or more targets.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### EXAMPLES

A sample in about 5 μl volume is mixed with the following in a final volume of 50 μl:
probe DNA at 1 μM
PCR primers, each at 100 nM
4 U Pfu polymerase
2 U Taq-ligase
1 mM NADP
2 mM dNTPs (each)
Tris buffer
2.0 mM MgSO₄

Thermocycling is performed using the following conditions described below. First cycle is performed in order anneal the probe to the target nucleic acid, extend and ligate to form a covalent circular target nucleic acid:
1 cycle of 95° C., 1 minute
   55° C. 30 sec
   65° C., 2 minutes.
      The extension is performed non strand-displacing conditions (e.g., at 65° C.). Then the following cycle is performed to detect the covalent circular target nucleic acid:
30 cycles of 95° C., 30 sec
   55° C. 30 sec
   72° C., 30 sec
      After the second cycling, the reaction products are analyzed by agarose gel electrophoresis.

1. A method for detecting a target nucleic acid, comprising:
a. hybridizing said target nucleic acid having a 5′ end and a 3′ end to a probe to form a circular hybridization complex, said probe having a 5′ end and a 3′ end, said probe comprising a first target nucleic acid binding site and a second target nucleic acid binding site, and wherein said probe is blocked from its 3′ end and not ligatable from its 5′ end;
b. forming a closed target nucleic acid using said probe as a template for nucleic acid synthesis; and
c. detecting said covalent circular target nucleic acid.

2. The method of claim 1, wherein said probe is between 50 and 300 bases in length.

3. The method of claim 1, wherein said probe is between 100 and 200 bases in length.

4. The method of claim 1, wherein said first target nucleic acid binding site is at one of said 5′ end or 3′ end of said probe.

5. The method of claim 4, wherein said second target nucleic acid binding site is at the other of said 5′ end or 3′ end of said probe.

6. The method of claim 1, wherein a DNA polymerase is used for said nucleic acid synthesis.

7. The method of claim 1, wherein said DNA polymerase is exonuclease deficient.

8. The method of claim 6, wherein a DNA polymerase is a non strand-displacing polymerase.

9. The method of claim 6, wherein said DNA polymerase is selected from the group consisting of T7-DNA polymerase, Pfu-DNA polymerase, Vent DNA polymerase.

10. The method of claim 1, wherein said first target nucleic acid binding site is between 10 and 40 bases in length.

11. The method of claim 1, wherein said first target nucleic acid binding site is between 15 and 25 bases in length.

12. The method of claim 1, wherein said second target nucleic acid binding site is between 10 and 40 bases in length.

13. The method of claim 1, wherein said second target nucleic acid binding site is between 15 and 25 bases in length.

14. The method of claim 1, wherein said 5′ end of said probe is dephosphorylated.

15. The method of claim 1, wherein said 3′ end nucleotide of said probe is a dideoxy nucleic acid.

16. The method of claim 1, wherein said probe further comprises at least one primer binding region.

17. The method of claim 1, wherein said probe further comprises a first primer binding region and a second primer binding region.

18. The method of claim 17, wherein said primer binding regions are located at least 15 bases from one of said target nucleic acid binding sites.

19. The method of claim 18, wherein said first primer binding region is located at least 15 bases from said first target nucleic acid binding site, and wherein said second primer binding site is located at least 15 bases from said second target nucleic acid binding site.

20. The method of claim 1, wherein said covalent target nucleic acid is detected by polymerase chain reaction.

21. A composition, comprising
a. A probe having a 5′ end and a 3′ end, said probe comprising a first target nucleic acid binding site and a second target nucleic acid binding site, and wherein said probe is blocked from its 3′ end and not ligatable from its 5′ end;
b. A polymerase;

22. The composition of claim 21, further comprising a target nucleic acid, having a 5′ end and a 3′ end.

23. The composition of claim 21, wherein said polymerase is a DNA polymerase.

24. The composition of claim 23, wherein said DNA polymerase is a non strand-displacing polymerase.

25. The composition of claim 23, wherein said DNA polymerase is exonuclease deficient.

26. The composition of claim 21, further comprising a primer.

27. The composition of claim 21, further comprising a ligase.

28. A kit, comprising:
a. A probe having a 5′ end and a 3′ end, said probe comprising a first target nucleic acid binding site and a second target nucleic acid binding site, and wherein said probe is blocked from its 3′ end and not ligatable from its 5′ end;
b. A polymerase;
c. packaging material therefor.

29. The kit of claim 28, further comprising a primer.

30. The kit of claim 28, wherein said polymerase is a DNA polymerase.

31. The kit of claim 30, wherein said DNA polymerase is a non strand-displacing polymerase.

32. The kit of claim 30, wherein said DNA polymerase is exonuclease deficient.

33. The kit of claim of claim 28, further comprising a ligase.
